(19) United States
(12) Patent Application Publication
(10) Pub. No.: US 2020/0062720 A1
(43) Pub. Date: Feb. 27, 2020

(54) NOVEL PREPARATION METHOD FOR ANTI-GOUT DRUG LESINURAD, AND KEY INTERMEDIATE THEREOF

(71) Applicants: Zhejiang Huahai Pharmaceutical Co., Ltd., Taizhou (CN); Shanghai Aobo Pharmtech, Inc., Ltd., Shanghai (CN)

(72) Inventors: Qing LI, Shanghai (CN); Fengmin XIA, Shanghai (CN); Chao HUANG, Shanghai (CN); Xiaowen GUO, Shanghai (CN); Anping TAO, Shanghai (CN); Luning HUANG, Shanghai (CN); Hong GU, Shanghai (CN)

(21) Appl. No.: 16/491,444

(22) PCT Filed: Jul. 11, 2018
(86) PCT No.: PCT/CN2018/095269

§ 371 Date: Sep. 5, 2019

(30) Foreign Application Priority Data
May 17, 2017 (CN) 201710346180.2

(51) Int. Cl. C07D 249/12

(52) U.S. Cl. CPC C07D 249/12

(57) ABSTRACT

A novel preparation method for the anti-gout drug Lesinurad, and a key intermediate thereof. The method comprises the following reaction steps: 1) the compound of formula II undergoing a substitution reaction with R₃—SH in the presence of a first solvent and a first alkali to generate a mixture containing the compound of formula III and the compound of formula IV; 2) adding a second alkali and R₃X to the resulting mixture for a reaction to obtain the compound of formula III, wherein: R represents a cyclopropane group, a halogen, a triflate group, a mesylate group or a tosylate group, preferably a cyclopropane group; R₃ represents —COCH₃, a benzyl group or —CH₂R₄, wherein R₄ represents a methyl acetate group, an ethyl acetate group, —C(O)OC₂H₅, —C(O)OCH₃, —CN, —CH₂OH or a phenyl group substituted with one or more of a C1-C6 alkyl group and a halogen; X represents a halogen. The process of the present invention directly converts the compound of formula IV into the product compound of formula III without separation, significantly increasing the reaction yield and simplifying the operation steps. In addition, the synthesis of the new intermediate of the present invention does not require the use of highly toxic thiophosgene and carbon disulphide, significantly improving the safety and environmental friendliness of the process.

The present application claims the priority of Chinese Patent Application No. 201710346180.2, filed before the CNIPA on May 17, 2017, titled “NOVEL PREPARATION METHOD FOR ANTI-GOUT DRUG LESINURAD, AND KEY INTERMEDIATE THEREOF”, which is incorporated herein by reference in its entirety.

### FIELD OF THE INVENTION

The invention relates to the technical field of drug synthesis, in particular to a novel preparation method of an anti-gout drug Lesinurad and a key intermediate thereof.

### BACKGROUND OF THE INVENTION

Gout is a crystal arthropathy caused by deposition of monosodium urate (MSU), which is directly related to hyperuricemia caused by a metabolic disorder of purine and/or a decrease in uric acid excretion. More than 20 million patients suffer from gout worldwide. Lesinurad is an oral SLC22A12 inhibitor, and SLC22A12 is also known as urate transporter 1 (URAT1) and organic anion transport protein 4 (OAT4). In December 2015, the European Medicines Agency (EMA) approved a drug from AstraZeneca, Lesinurad, in combination with another xanthine oxidase inhibitor which reduces in vivo uric acid production as a treatment for gout-associated hyperuricemia.

The following literatures reported the synthetic routes of the Lesinurad compound:

(1) The synthetic route reported in patent WO2006026356 is as follows:

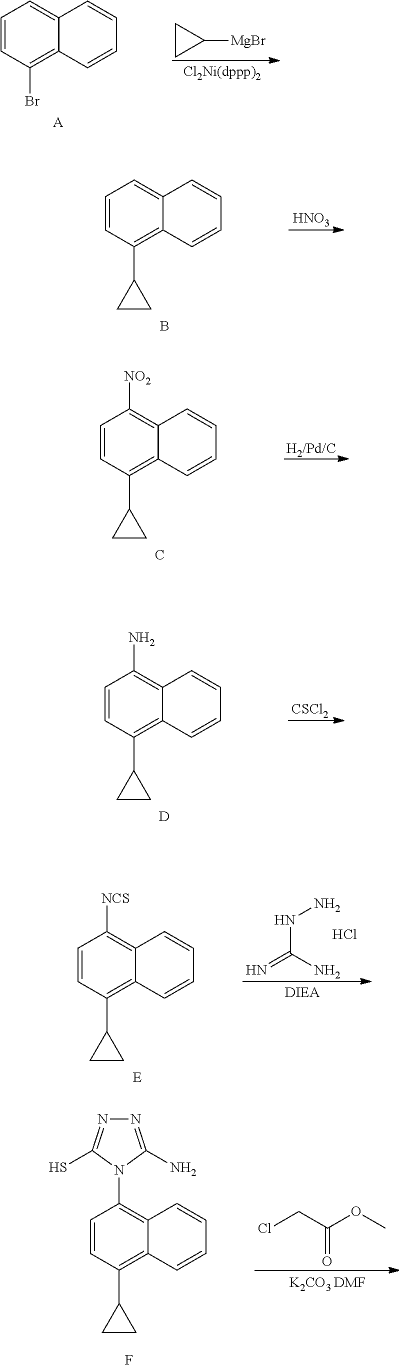

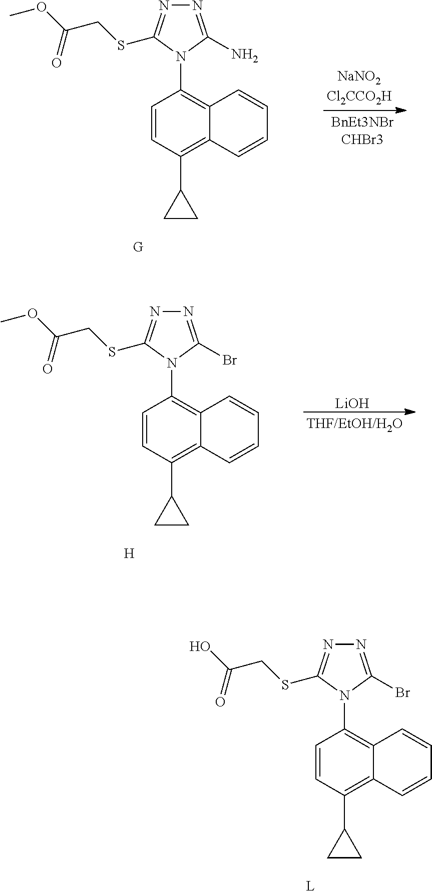

The route is a synthetic route in the patent of compound Lesinurad reported by the original research company with lengthy reaction procedure. When the compound D is calculated as the starting material, the total yield is about 25.8%. The route uses highly toxic thiophosgene, which has certain impact on environment, health and safety.

(2) The synthetic route reported in patent WO2014008295 is as follows:

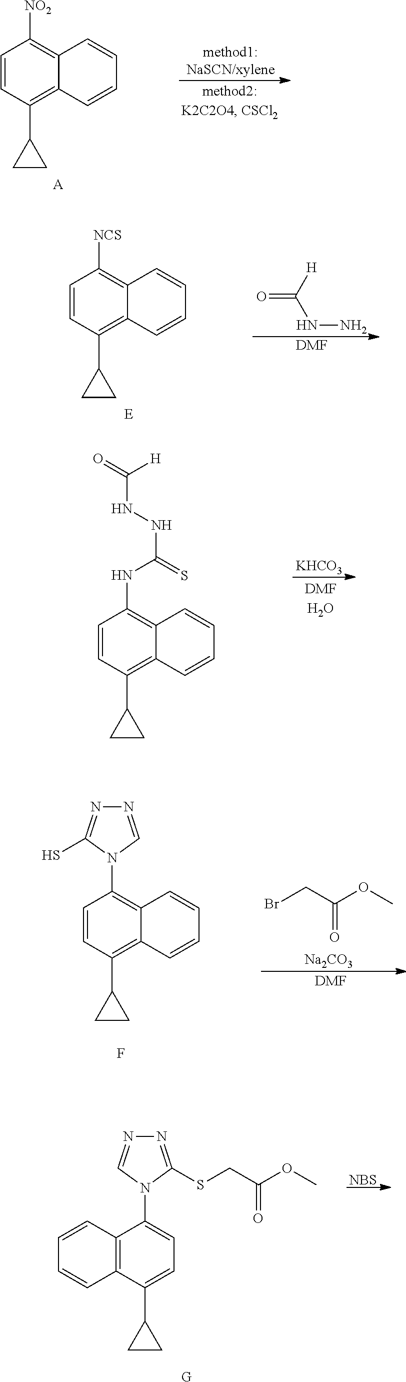

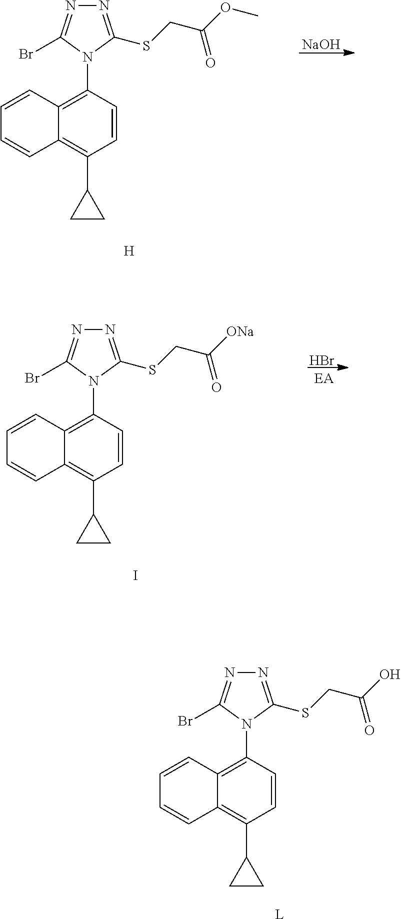

The route is used in the patent for the preparation of Lesinurad of the original research company with good total yield, but the highly toxic thiophosgene is used in the route.

(3) The synthetic route reported in Chinese patent CN102040546 is as follows:

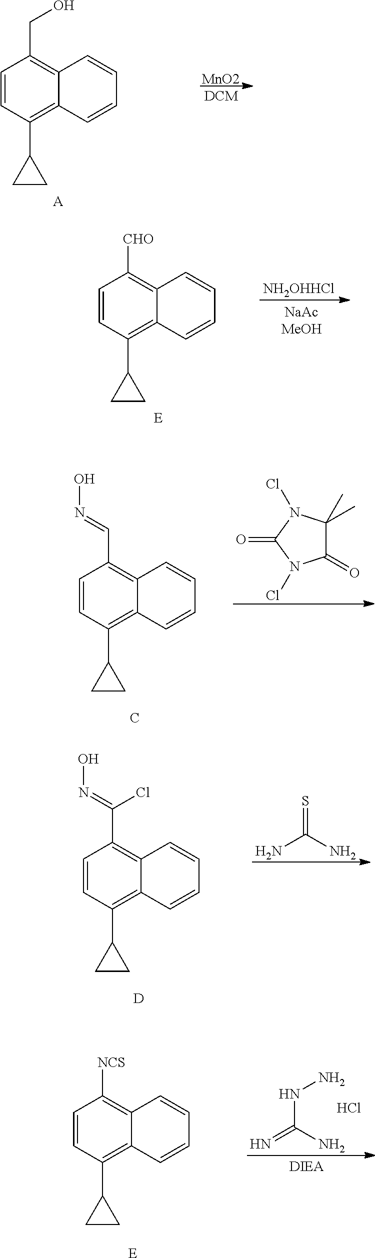

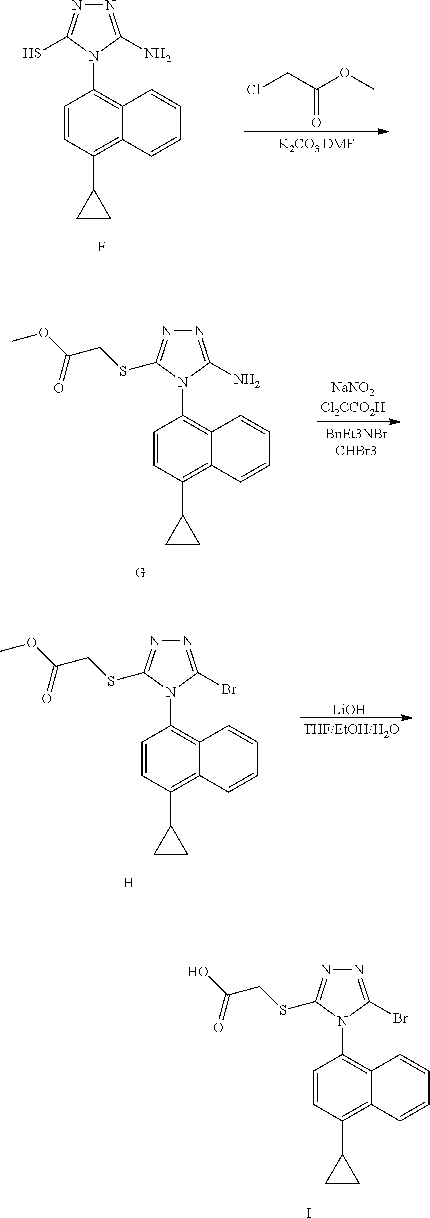

Although the route avoids the use of thiophosgene which is harmful to the environment, health and safety, it has disadvantages that the starting materials used are hard to obtain and expensive, and the total yield is only about 25%.

(4) The synthetic route reported in Chinese patent CN103524440 is as follows:

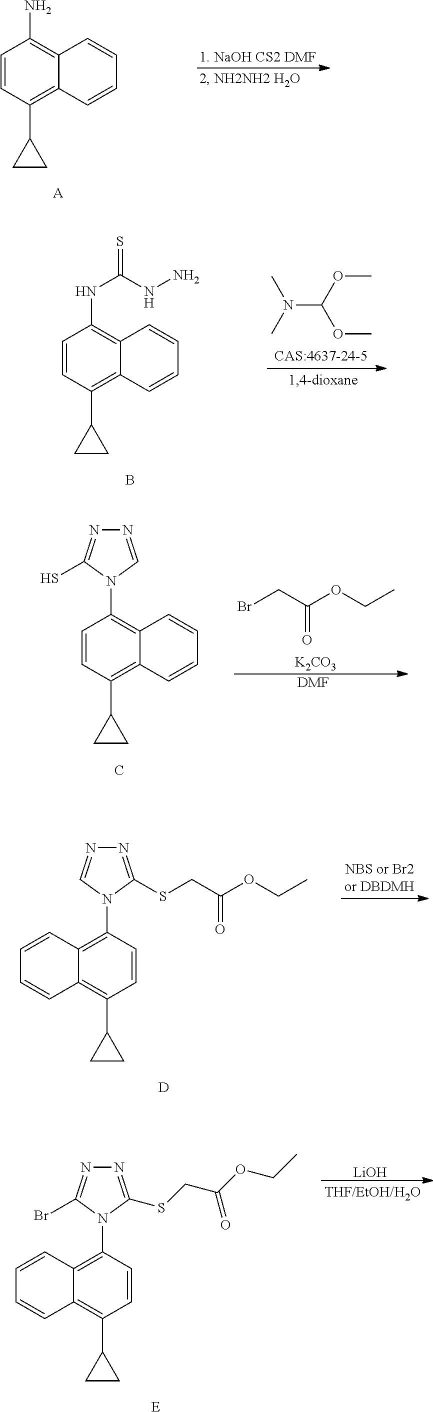

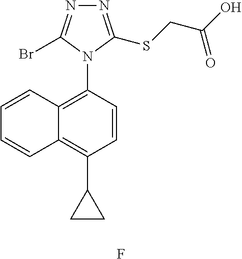

The route is similar to the preparation route of the original research company. The mercaptotriazole ring is obtained by cyclization of different hydrazines reagents, and then bromination followed by hydrolysis to obtain Lesinurad. However, the route has a long reaction procedure and the highly toxic carbon disulfide is used in the route. The process requires purification by column chromatography in the bromination step, which is complicated for operation and is not suitable for industrial production.

A comprehensive analysis of the existing preparation methods of Lesinurad reveals that most of the bromine in the Lesinurad structure is converted from amino groups. This step is complicated for operation and the starting materials or reagents used are expensive, resulting in high production cost. In addition, most of the existing preparation methods involve the use of highly toxic thiophosgene or carbon disulfide, which cause many disadvantages in the safety, economical and large-scale production of the reaction.

### SUMMARY OF THE INVENTION

The technical problem to be solved in the present invention is to overcome the deficiencies of the prior art by providing a novel preparation method of Lesinurad and a new intermediate thereof. The method is a synthetic process which is more economical, more efficient, safer, more environmentally friendly, and suitable for large-scale industrial production.

The invention is achieved by the following technical solution: a preparation method of a Lesinurad intermediate compound of the formula III, and the specific synthetic route is as follows:

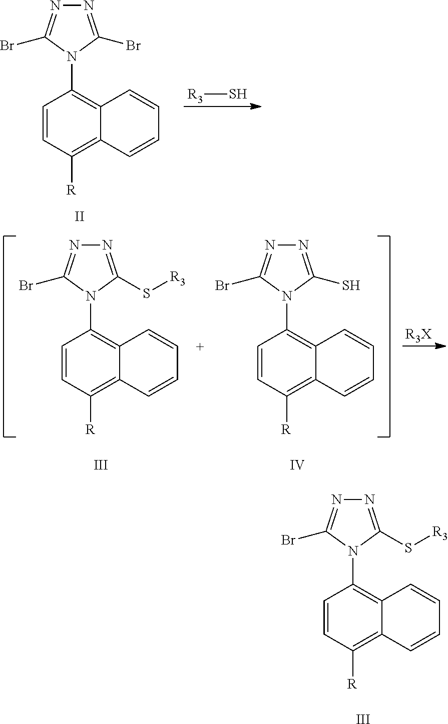

The method comprises the following steps: subjecting a compound of formula II and R₃—SH to a substitution reaction in the presence of a first solvent and a first base to form a mixture comprising a compound of formula III and a compound of formula IV; and adding a second base and R₃X to the mixture for reaction to obtain a compound of formula III;

wherein R represents cyclopropyl, halogen, triflate group, mesylate group, tosylate group, preferably, R represents cyclopropyl;
R₃ represents —COCH₃, benzyl or —CH₂R₄, wherein R₄ represents an ester group, —C(O)OC₂H₅, —C(O)OCH₃, —CN, —CH₂OH or a phenyl substituted with one or more C₁-C₆ alkyl or halogen; and
X represents a halogen.

According to the preparation method provided by the present invention, the first solvent is selected from the group consisting of N,N-dimethylformamide, N-methylpyrrolidone and acetonitrile, or any combination thereof.

According to the preparation method provided by the present invention, the first base and the second base in the step 1) and the step 2) are independently selected from the group consisting of 1,8-diazabicycloundec-7-ene, diisopropylethylamine, triethylamine, potassium carbonate and sodium carbonate, or any combination thereof. The first base and the second base may be the same or different.

According to the preparation method provided by the present invention, the mixture obtained in the step 1) can be directly used in the next step for converting into the compound III without further purification.

The inventors have found through research that in the preparation of lesinurad, the substitution reaction of the compound of formula II with R₃—SH in the presence of the first solvent and the first base will inevitably produce a mixture containing the compound of formula III and the compound of formula IV, wherein the compound of formula III accounts for about 50%, and the compound of formula IV accounts for about 30%. If the compound of formula IV is directly removed as an impurity, the yield will be lowered and the cost will be affected. If the crude product of the compound of formula III containing a large amount of the compound of formula IV is directly subjected to the next hydrolysis, it will result in a large content of the impurities in the crude final product, which is difficult to purify. In addition, the compound of formula III and the compound of formula IV have little difference in polarity, and their contents in the mixture are not much different, thus it is difficult to purify by crystallization or slurrying. The inventors have surprisingly found that after treating with R₃X, almost all of the compound of formula IV is converted to the compound of formula III, which greatly increasing the yield of the compound of formula III, and the subsequent reaction can be carried out directly without purification to finally form Lesinurad.

In some specific embodiments of the present invention, when R is cyclopropyl, the compound of formula III can be converted to Lesinurad by a further group conversion reaction of R₃ in the compound of formula III, such as a hydrolysis reaction.

In other specific embodiments of the present invention, when R represents halogen, triflate group, mesylate group or tosylate group, R can be converted to cyclopropyl by subjecting to a Grignard reaction or a hydrolysis reaction followed by a Grignard reaction, or other conventional chemical reactions;

and finally converting the compound of formula III into Lesinurad by subjecting R₃ in the compound of formula III to a further group conversion reaction, such as a hydrolysis reaction.

It should be noted that, in this specific embodiment, there is no specific requirement for the sequential order between the reaction of converting R into cyclopropyl and the group conversion reaction of R₃. That is either the former may be carried out first, or the latter may be carried out first. In another aspect, the present invention provides Lesinurad intermediate compounds of the formula I and formula II, the structural formulas of which are as follows:

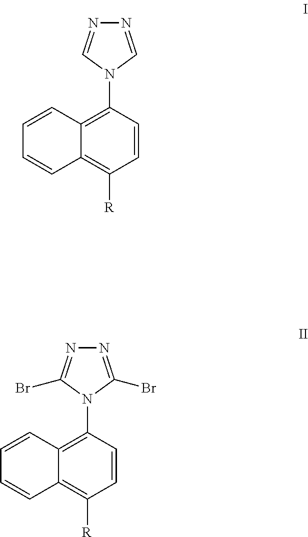

wherein, R represents cyclopropyl, halogen, triflate group, mesylate group or tosylate group; preferably, R represents cyclopropyl. In some embodiments of the invention, the compounds of the formula I and formula II are more specifically compound 2 and compound 3 of the following formulas:

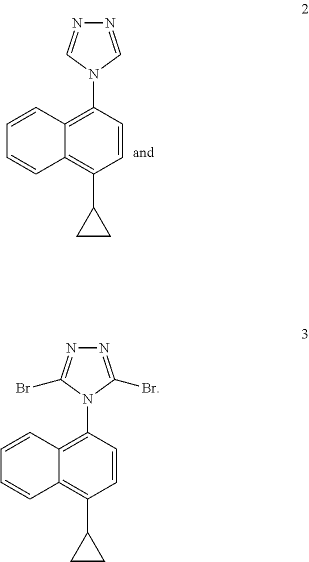 [SMILES: c1ccc2c(-n3cnnc3)ccc(C3CC3)c2c1 ; Brc1nnc(Br)n1-c1ccc(C2CC2)c2ccccc12]

Another technical solution of the present invention is a method for preparing a Lesinurad intermediate compound of the formula II, which comprises subjecting a compound of formula I to a bromination reaction in a second solvent to form a compound of formula II. The reaction is as follows:

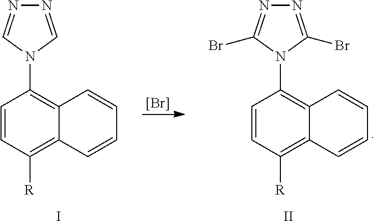

According to the preparation method provided by the present invention, the bromination reagent used in the bromination reaction is selected from the group consisting of liquid bromine, bromine water, N-bromosuccinimide, dibromohydantoin, ammonium phenyltrimethyltribromide, 5,5-dibromobarbituric acid and dibromoisocyanuric acid, or any combination thereof; the second solvent is selected from the group consisting of tetrahydrofuran, 2-methyltetrahydrofuran, dichloromethane and acetonitrile, or any combination thereof.

The Lesinurad intermediate compound of formula I can be obtained by reacting a compound of formula V or a salt thereof and N,N-diformylhydrazine in a third solvent in the presence of trimethylhalosilane and a third base. The reaction is as follows:

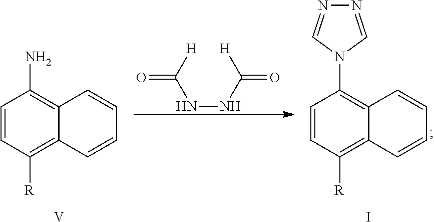

wherein, R represents cyclopropyl, halogen, triflate group, mesylate group or tosylate group; preferably, R represents cyclopropyl.

The third solvent described in the preparation method provided by the present invention is selected from the group consisting of pyridine, acetonitrile and toluene, or any combination thereof. The third base is selected from the group consisting of pyridine, triethylamine and diisopropylethylamine (DIPEA), or any combination thereof. The trimethylhalosilane is selected from the group consisting of trimethylchlorosilane, trimethylbromosilane, and trimethyliodosilane, or any combination thereof.

A more specific technical solution of the present invention is a preparation method of Lesinurad compound, and the preparation procedure is as follows:

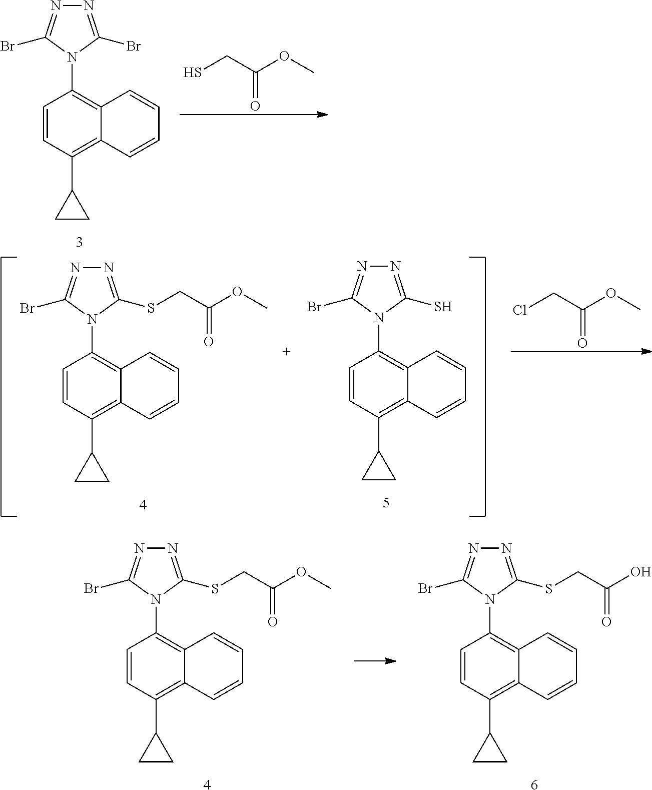

The method comprises: subjecting compound 3 and methyl mercaptoacetate to a substitution reaction in the presence of a first solvent and a first base to form a mixture containing compound 4 and compound 5; and adding a second base and methyl chloroacetate to the mixture for reaction to obtain compound 4; further converting compound 4 to lesinurad, i.e. compound 6; preferably, directly converting compound 4 obtained in step 2) to lesinurad by a further hydrolysis without purification;

wherein the first solvent is selected from the group consisting of N,N-dimethylformamide, N-methylpyrrolidone and acetonitrile, or any combination thereof; the first base and the second base of the steps 1) and 2) are independently selected from the group consisting of 1,8-diazabicycloundec-7-ene, diisopropylethylamine, triethylamine, potassium carbonate and sodium carbonate, or any combination thereof.

Compared with the prior art, the technical solution provided by the present invention has the following beneficial technical effects:

(1) A new lesinurad intermediate and a novel preparation method thereof are provided, which make the process possible to avoid the use of thiophosgene and carbon disulfide, wherein thiophosgene is highly toxic and complicated for operation and carbon disulfide is toxic to damage nerve and vascular.
(2) An efficient preparation method for lesinurad is provided, which is advantageous for quality control, high conversion rate, and low production cost.
(3) According to the method of the present invention, in the synthesis of the compound of formula III from the compound of formula II, the product is obtained by a two-stage reaction without isolation and purification, and the crude product containing the compound of formula III can be used for the next reaction, which is simple and convenient in operation and conducive to production capacity and industrial production.
(4) The total yield of the reaction is high, compared with other routes in the prior art. The reaction yield can be increased from about 25% to about 44%.

Of course, any method of implementing the invention does not necessarily achieve all of the advantages described above at the same time.

### DESCRIPTION OF THE EMBODIMENT

In order to make the objects, technical solutions, and advantages of the present invention more clear and comprehensible, the present invention will be further described in detail below through specific examples. It is apparent that the described examples are only a part of the examples of the invention, not all of the examples. All other examples obtained by those skilled in the art based on the examples of the present invention without creative efforts are within the scope of the present invention.

In a specific embodiment of the present invention, the preparation method of Lesinurad can be expressed by the reaction equation as follows:

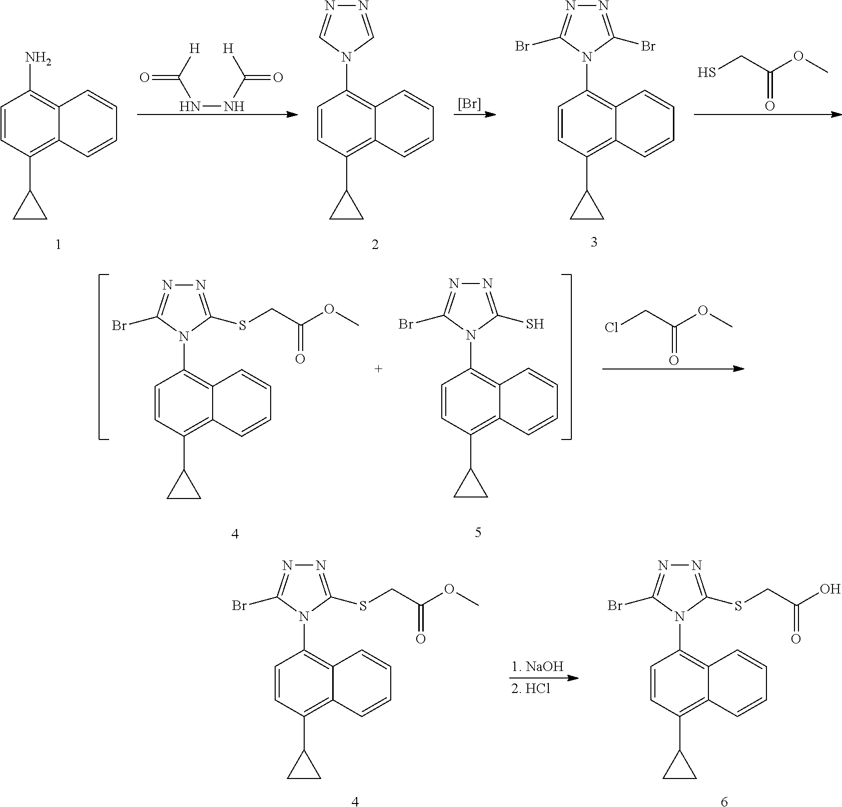

The present invention is further illustrated by the following examples. However, these examples are not intended to limit the present invention.

### Example 1: Preparation of 4-(4-cyclopropylnaphthalene)-1,2,4-triazole

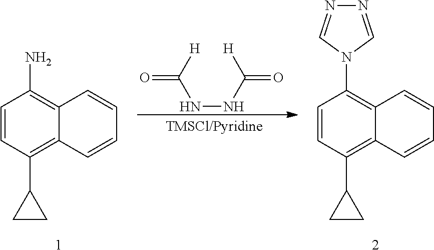

To a three-necked flask, 4-cyclopropyl-1-naphthylamine (compound 1, 20.00 g, 110.00 mmol), diformylhydrazine (29.06 g, 330.00 mmol) and pyridine (10 V, 200.00 ml) were added, and trimethylchlorosilane (59.75 g, 550.00 mmol) was slowly added dropwise at room temperature, and the reaction was then heated to reflux for 2 hours. After confirming the completion of the reaction by LC, the insoluble solid salt was removed by filtration, and the filtrate was concentrated to dryness. The obtained residue was dissolved in ethyl acetate. The organic phase was washed twice with water, dried and concentrated under reduced pressure to get about 30 ml of concentrate. 90 ml of methyl tert-butyl ether was added to the concentrate, and the resulting suspension was slurried and stirred for 1 hour, and subjected to suction filtration to obtain compound 2 (purity: 98%), yield (70%).

¹H NMR (400 MHz, CDCl₃) δ 8.56 (d, J=8.4 Hz, 1H), 8.41 (s, 2H), 7.70-7.66 (m, 1H), 7.60-7.56 (m, 1H), 7.44 (d, J=8.4 Hz, 1H), 7.38 (d, 7.6 Hz, 1H), 7.36 (d, 7.6 Hz, 1H), 2.44-2.40 (m, 1H), 1.20-1.15 (m, 2H), 0.86-0.82 (m, 2H); MS (ESI) m/z 236.11 ([M+H]⁺).

### Example 2: Preparation of 4-(4-cyclopropylnaphthalene)-1,2,4-triazole

To a three-necked flask, 4-cyclopropyl-1-naphthylamine (compound 1, 9.16 g, 50.00 mmol), diformylhydrazine (14.53 g, 165.00 mmol), toluene (10 V, 91.60 ml) and pyridine (15.82 g, 200.00 mmol) were added, and trimethylchlorosilane (29.87 g, 275.00 mmol) was slowly added dropwise at room temperature, and the reaction was then heated to reflux for 2 hours. After confirming the completion of the reaction by LC, the insoluble solid salt was removed by filtration, and the filtrate was concentrated to dryness. The obtained residue was dissolved in ethyl acetate. The organic phase was washed twice with water, dried and concentrated under reduced pressure to get about 15 ml of concentrate. 45 ml of methyl tert-butyl ether was added to the concentrate, and the resulting suspension was slurried and stirred for 1 hour, and subjected to suction filtration to obtain compound 2 (purity: 98.2%), yield (78%).

### Example 3: Preparation of 4-(4-cyclopropylnaphthalene)-1,2,4-triazole

To a three-necked flask, 4-cyclopropyl-1-naphthylamine (compound 1, 9.16 g, 50.00 mmol), diformylhydrazine (14.53 g, 165.00 mmol), acetonitrile (10 V, 91.6 ml) and triethylamine (20.24 g, 200 mmol) were added, and trimethylbromosilane (29.87 g, 275 mmol) was slowly added dropwise at room temperature, and the reaction was then heated to reflux for 2 hours. After confirming the completion of the reaction by LC, the insoluble solid salt was removed by filtration, and the filtrate was concentrated to dryness. The obtained residue was dissolved in ethyl acetate. The organic phase was washed twice with water, dried and concentrated under reduced pressure to get about 15 ml of concentrate. 45 ml of methyl tert-butyl ether was added to the concentrate, and the resulting suspension was slurried and stirred for 1 hour, and subjected to suction filtration to obtain compound 2 (purity: 98.0%), yield (77%).

### Example 4: Preparation of 4-(4-cyclopropylnaphthalene)-3,5-dibromo-1,2,4-triazole

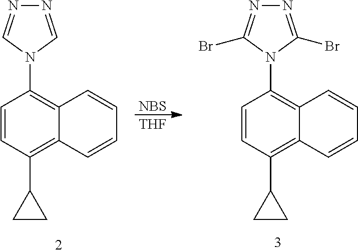

To a three-necked flask, 4-(4-cyclopropylnaphthalene)-1,2,4-triazole (compound 2, 11.50 g, 48.91 mmol) and tetrahydrofuran (6 V, 69.00 ml) were added, and N-bromosuccinimide (34.96 g, 122.28 mmol) was added in batches at room temperature. The reaction was then stirred at 40° C. for 2 hours. After the end of the reaction confirmed by LC, the reaction solution was diluted with ethyl acetate. The organic phase was washed twice with 30% sodium thiosulfate solution and saturated sodium bicarbonate solution respectively, dried and concentrated. 40 ml of methyl tert-butyl ether was added to the residue. The suspension was stirred and slurried for 1 hour, and subjected to suction filtration. The filter cake was washed twice with 10 ml methyl tert-butyl ether to obtain compound 3 (purity: 99%), yield (85%).

¹H NMR (400 MHz, CDCl₃) δ 8.58 (d, J=8.4 Hz, 1H), 7.71-7.67 (m, 1H), 7.62-7.58 (m, 1H), 7.41 (d, 7.6 Hz, 1H), 7.35 (d, 7.6 Hz, H), 7.18 (d, J=8.4 Hz, 1H), 2.47-2.44 (m, 1H), 1.21-1.18 (m, 2H), 0.92-0.88 (m, 2H); MS (ESI) m/z 391.93 ([M+H]⁺).

### Example 5: Preparation of 4-(4-cyclopropylnaphthalene)-3,5-dibromo-1,2,4-triazole

To a three-necked flask, 4-(4-cyclopropylnaphthalene)-1,2,4-triazole (compound 2, 11.50 g, 48.91 mmol) and dichloromethane (6 V, 69.00 ml) were added, and liquid bromine (34.96 g, 122.28 mmol) was added in batches at room temperature. After that, the reaction was then stirred at 40° C. for 2 hours. After the end of the reaction confirmed by LC, the reaction solution was diluted with ethyl acetate. The organic phase was washed twice with 30% sodium thiosulfate solution and saturated sodium bicarbonate solution respectively, dried and concentrated. 40 ml of methyl tert-butyl ether was added to the residue. The suspension was stirred and slurried for 1 hour, and subjected to suction filtration. The filter cake was washed twice with 10 ml methyl tert-butyl ether to obtain compound 3 (purity: 98%), yield (83%). MS (ESI) m/z 391.93 ([M+H]⁺).

### Example 6: Preparation of 4-(4-cyclopropylnaphthalene)-3,5-dibromo-1,2,4-triazole

To a three-necked flask, 4-(4-cyclopropylnaphthalene)-1,2,4-triazole (compound 2, 11.50 g, 48.91 mmol) and tetrahydrofuran (6 V, 69.00 ml) were added, and dibromohydantoin (34.96 g, 122.28 mmol) was added in batches at room temperature. After that, the reaction was then stirred at 40° C. for 2 hours. After the end of the reaction confirmed by LC, the reaction solution was diluted with ethyl acetate. The organic layer was washed twice with 30% sodium thiosulfate solution and saturated sodium bicarbonate solution respectively, dried and concentrated. 40 ml of methyl tert-butyl ether was added to the residue. The suspension was stirred and slurried for 1 hour, and subjected to suction filtration. The filter cake was washed twice with 10 ml methyl tert-butyl ether to obtain compound 3 (purity: 98%), yield (82%). MS (ESI) m/z 391.93 ([M+H]⁺).

### Example 7A: Preparation of 4-(4-cyclopropylnaphthalene)-3-methyl thioacetate-5-dibromo-1,2,4-triazole

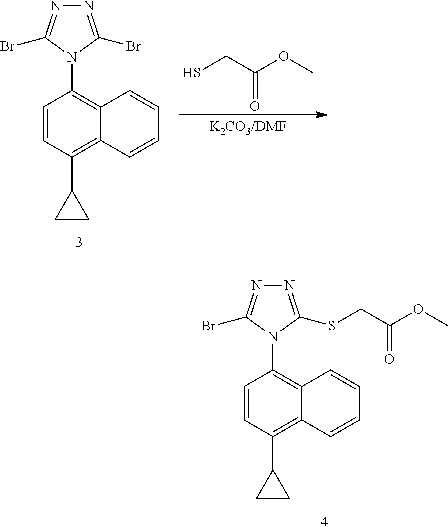

To a three-necked flask, 4-(4-cyclopropylnaphthalene)-3,5-dibromo-1,2,4-triazole (compound 3, 4.00 g, 10.18 mmol) and N,N-dimethylformamide (10 V, 40.00 ml) were added, and potassium carbonate (2.10 g, 15.26 mmol) and methyl mercaptoacetate (1.62 g, 15.26 mmol) were successively added at room temperature. The reaction was stirred at room temperature for 1 hour, and the depletion of the starting material was confirmed by LC. The reaction mixture was diluted with ethyl acetate. The organic phase was washed once with 0.5N hydrochloric acid solution, and then washed three times with water. A crude product of compound 4 was obtained by drying and concentrating. It was purified through silica gel column to obtain compound 4 (purity: 90%), yield (50%)

¹H NMR (400 MHz, CDCl₃) δ 8.55 (d, J=8.4 Hz, 1H), 7.68-7.64 (m, 1H), 7.60-7.56 (m, 1H) 7.36 (s, 2H), 7.26 (d, J=8.4 Hz, 1H), 4.09 (d, J 16.4 Hz, 1H), 4.03 (d, J=16.4 Hz, 1H), 3.72 (s, 3H), 2.45-2.41 (m, 1H), 1.19-1.15 (m, 2H), 0.90-0.86 (m, 2H); MS (ESI) m/z 418.01 ([M+H]⁺).

### Example 7B: Preparation of 4-(4-cyclopropylnaphthalene)-3-methyl thioacetate-5-dibromo-1,2,4-triazole

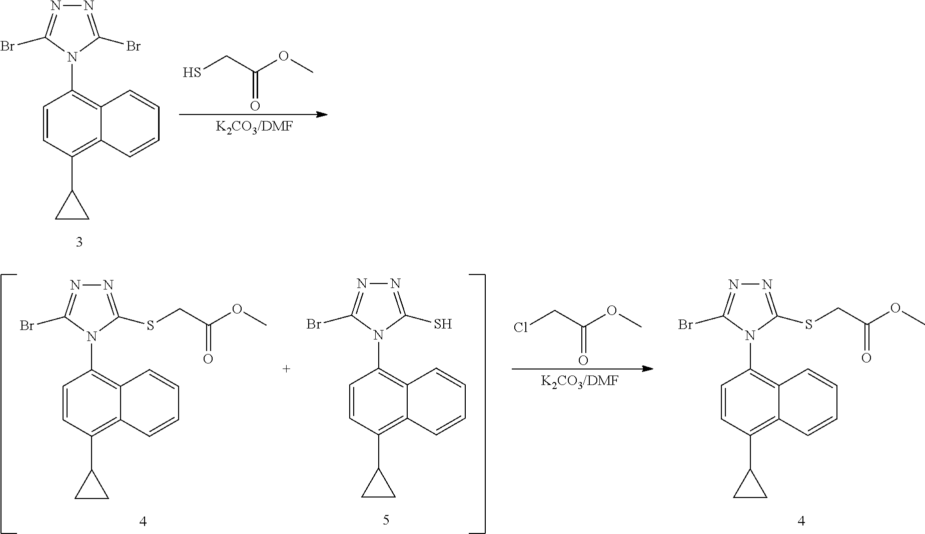

To a three-necked flask, 4-(4-cyclopropylnaphthalene)-3,5-dibromo-1,2,4-triazole (compound 3, 4.64 g, 11.80 mmol) and N,N-dimethylformamide (10 V, 46.40 ml) were added, and potassium carbonate (2.45 g, 17.71 mmol) and methyl mercaptoacetate (1.88 g, 17.71 mmol) were successively added at room temperature. The reaction was stirred at room temperature for 1 hour, and the depletion of the starting material was confirmed by LC. At this time, potassium carbonate (1.79 g, 12.98 mmol) and methyl chloroacetate (1.41 g, 12.98 mmol) were successively added to the reaction system, and stirring continued for 1 hour at room temperature. After completion of the reaction, the reaction mixture was diluted with ethyl acetate. The organic phase was washed once with 0.5N hydrochloric acid solution, and then washed three times with water. A crude product of compound 4 was obtained by drying and concentrating. It was used for the next reaction without purification.

### Example 8: Preparation of 4-(4-cyclopropylnaphthalene)-3-methyl thioacetate-5-dibromo-1,2,4-triazole

To a three-necked flask, 4-(4-cyclopropylnaphthalene)-3,5-dibromo-1,2,4-triazole (compound 3, 4.64 g, 11.80 mmol) and acetonitrile (10 V, 46.40 ml) were added, and potassium carbonate (1.79 g, 17.71 mmol) and methyl mercaptoacetate (1.88 g, 17.71 mmol) were successively added at room temperature. The reaction was stirred at room temperature for 1 hour, and the depletion of the starting material was confirmed by LC. At this time, sodium carbonate (1.37 g, 12.98 mmol) and methyl chloroacetate (1.41 g, 12.98 mmol) were successively added to the reaction system, and stirring continued for 1 hour at room temperature. After completion of the reaction, the reaction mixture was diluted with ethyl acetate. The organic phase was washed once with 0.5N hydrochloric acid solution, and then washed three times with water. A crude product of compound 4 was obtained by drying and concentrating. It was used for the next reaction without purification.

### Example 9: Preparation of 4-(4-cyclopropylnaphthalene)-3-methyl thioacetate-5-dibromo-1,2,4-triazole

To a three-necked flask, 4-(4-cyclopropylnaphthalene)-3,5-dibromo-1,2,4-triazole (compound 3, 4.64 g, 11.80 mmol) and N-methylpyrrolidone (10 V, 46.40 ml) were added, and potassium carbonate (2.45 g, 17.71 mmol) and methyl mercaptoacetate (1.88 g, 17.71 mmol) were successively added at room temperature. The reaction was stirred at room temperature for 1 hour, and the depletion of the starting material was confirmed by LC. At this time, potassium carbonate (1.79 g, 12.98 mmol) and methyl chloroacetate (1.41 g, 12.98 mmol) were successively added to the reaction system, and stirring continued for 1 hour at room temperature. After completion of the reaction, the reaction mixture was diluted with ethyl acetate. The organic phase was washed once with 0.5N hydrochloric acid solution, and then washed three times with water. A crude product of compound 4 was obtained by drying and concentrating. It was used for the next reaction without purification.

### Example 10: Preparation of 4-(4-cyclopropylnaphthalene)-3-ethyl thioacetate-5-dibromo-1,2,4-triazole

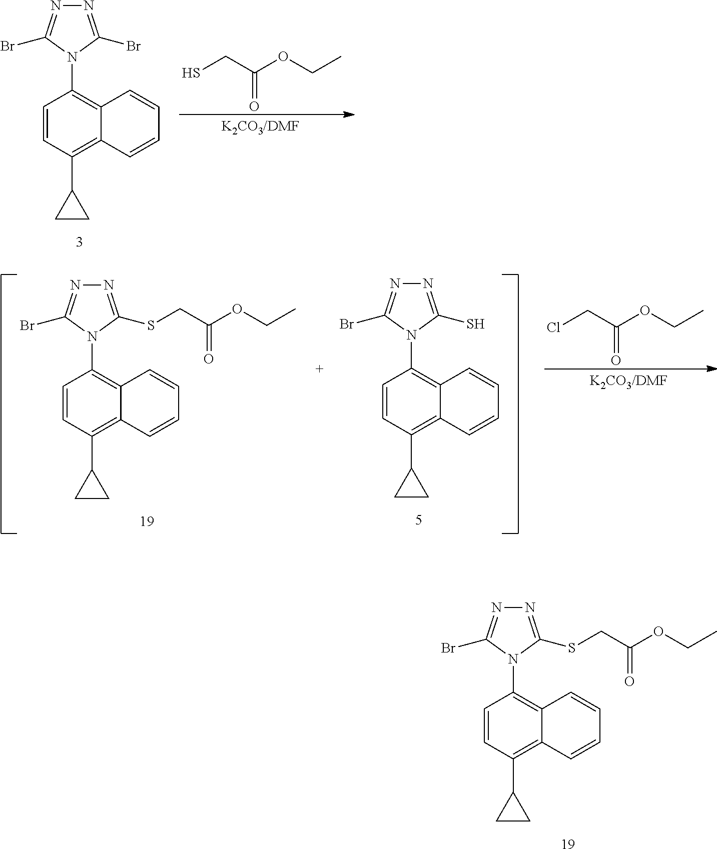

To a three-necked flask, 4-(4-cyclopropylnaphthalene)-3,5-dibromo-1,2,4-triazole (compound 3, 4.64 g, 11.80 mmol) and N,N-dimethylformamide (10 V, 46.40 ml) were added, and potassium carbonate (2.45 g, 17.71 mmol) and ethyl mercaptoacetate (2.13 g, 17.71 mmol) were successively added at room temperature. The reaction was stirred at room temperature for 1 hour, and the depletion of the starting material was confirmed by LC. At this time, potassium carbonate (1.79 g, 12.98 mmol) and ethyl chloroacetate (1.59 g, 12.98 mmol) were successively added to the reaction system, and stirring continued for 1 hour at room temperature. After completion of the reaction, the reaction mixture was diluted with ethyl acetate. The organic phase was washed once with 0.5N hydrochloric acid solution, and then washed three times with water. A crude product of compound 19 was obtained by drying and concentrating. It was used for the next reaction without purification (purity 89%, yield 79%).

¹H NMR (400 MHz, CDCl₃) δ 8.55 (d, J=8.4 Hz, 1H), 7.68-7.64 (m, 1H), 7.60-7.56 (m, 1H), 7.36 (s, 2H), 7.26 (d, J=8.4 Hz, 1H), 4.09 (d, J=16.4 Hz, 1H), 4.03 (d, J=16.4 Hz, 1H), 3.72 (s, 3H), 2.45-2.41 (m, 1H), 1.33-1.27 (t, 3H), 1.19-1.15 (m, 2H), 0.90-0.86 (m, 2H); MS (ESI) m/z 432.03 ([M+H]⁺).

### Example 11: Preparation of Lesinurad

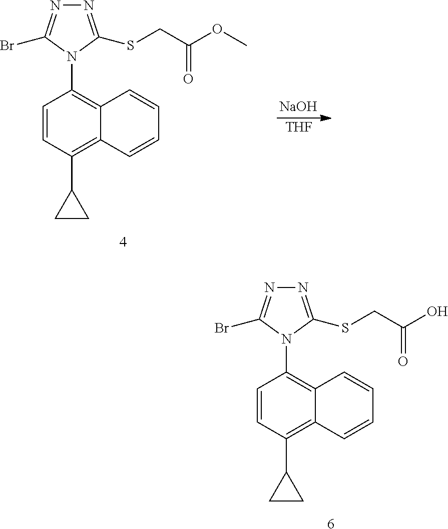

To a three-necked flask, 4-(4-cyclopropylnaphthalene)-3-methyl thioacetate-5-bromo-1,2,4-triazole (compound 4 without purification in the previous step, 4.93 g, 11.80 mmol) and tetrahydrofuran (10V, 49.30 ml) were added, and IN sodium hydroxide solution (23.60 ml, 23.60 mmol) was slowly added dropwise to the solution at room temperature. The mixture was stirred at room temperature for 2 hours. After the end of the reaction confirmed by LC, the reaction mixture was diluted with water. After the aqueous phase was washed twice with ethyl acetate, it was adjusted to acidity by adding IN hydrochloric acid solution. The aqueous phase was further extracted with ethyl acetate twice. The resulting organic phase was dried and concentrated to dryness to obtain compound 6 as white solid (purity: 98%) in two steps (yield 75%).

¹H NMR (400 MHz, CDCl₃) δ 8.57 (d, J=8.4 Hz, 1H), 8.26 (bs, 1H), 7.70-7.66 (m, 1H), 7.62-7.58 (m, 1H), 7.38 (s, 2H), 7.23 (d, J=8.4 Hz, 1H), 4.03 (d, J=15.6 Hz, 1H), 3.96 (d, J=15.6 Hz, 1H), 2.47-2.43 (m, 1H), 1.22-1.17 (m, 2H), 0.91-0.87 (m, 2H); MS (ESI) m/z 404.00 ([M+H]⁺).

The above are only the preferred examples of the present invention, which are not intended to limit the present invention. Any modifications, equivalents, improvements, etc., which are made within the spirit and principles of the present invention, should be included within the scope of the present invention.

1. A preparation method for a Lesinurad intermediate compound of formula III, 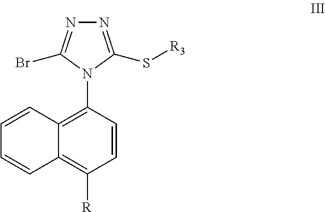
comprising:
1) subjecting a compound of formula II and R₃—SH to a substitution reaction in the presence of a first solvent and a first base to form a mixture comprising a compound of formula III and a compound of formula IV; and
2) adding a second base and R₃X to the mixture for reaction to obtain the compound of formula III; 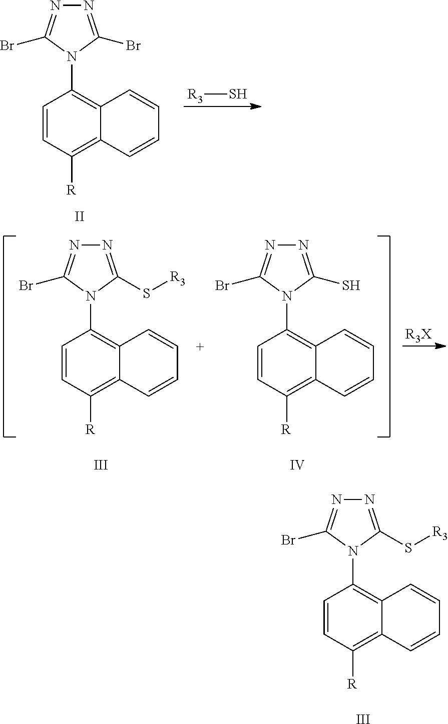
wherein, R represents cyclopropyl, halogen, triflate group, mesylate group or tosylate group;
R₃ represents —COCH₃, benzyl or —CH₂R₄, wherein R₄ represents methoxy carbonyl methylene, ethoxy carbonyl methylene, —C(O)OC₂H₅, —C(O)OCH₃, —CN, —CH₂OH or a phenyl substituted with one or more C₁-C₆ alkyl or halogen; and
X represents halogen.

2. The preparation method according to claim 1, wherein the first solvent is selected from the group consisting of N,N-dimethylformamide, N-methylpyrrolidone and acetonitrile, or any combination thereof; the first base in the step 1) and the second base in the step 2) are independently selected from the group consisting of 1,8-diazabicycloundec-7-ene, diisopropylethylamine, triethylamine, potassium carbonate and sodium carbonate, or any combination thereof.

3. The preparation method according to claim 1, wherein the mixture obtained in the step 1) is subjected to a next reaction directly without purification and is converted into the compound of formula III.

4. A Lesinurad intermediate compound of formula I or formula II, 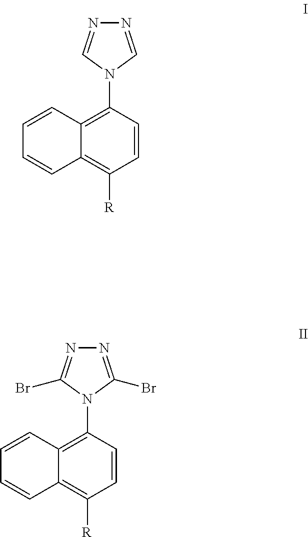
wherein, R represents cyclopropyl, halogen, triflate group, mesylate group or tosylate group.

5. A preparation method of the Lesinurad intermediate compound of formula II according to claim 4, comprising subjecting a compound of formula I to a bromination reaction in a second solvent to form the compound of formula II

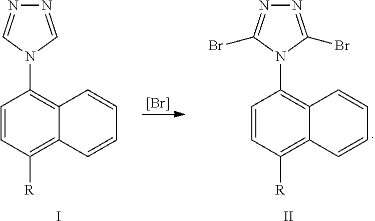

6. The preparation method according to claim 5, wherein a bromination reagent used in the bromination reaction is selected from the group consisting of liquid bromine, bromine water, N-bromosuccinimide, dibromohydantoin, ammonium phenyltrimethyltribromide, 5,5-dibromobarbituric acid and dibromoisocyanuric acid, or any combination thereof; the second solvent is selected from the group consisting of tetrahydrofuran, 2-methyltetrahydrofuran, dichloromethane and acetonitrile, or any combination thereof.

7. A preparation method of the Lesinurad intermediate compound of formula I according to claim 4, comprising subjecting a compound of formula V or a salt thereof and N,N-diformylhydrazine to a reaction in a third solvent in the presence of trimethylhalosilane and a third base to obtain the compound of formula I

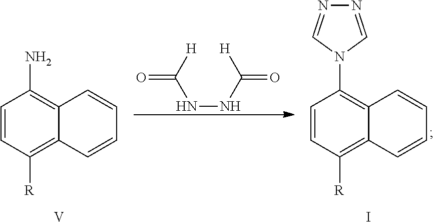

8. The preparation method according to claim 7, wherein the third solvent is selected from the group consisting of pyridine, acetonitrile and toluene, or any combination thereof; the third base is selected from the group consisting of pyridine, triethylamine and diisopropylethylamine, or any combination thereof, the trimethylhalosilane is selected from the group consisting of trimethylchlorosilane, trimethylbromosilane, and trimethyliodosilane, or any combination thereof.

9. A preparation method of the Lesinurad compound, comprising:
1) subjecting a compound 3 and methyl mercaptoacetate to a substitution reaction in the presence of a first solvent and a first base to form a mixture comprising a compound 4 and a compound 5;
2) adding a second base and methyl chloroacetate to the mixture for reaction to obtain the compound 4; and
3) converting the compound 4 to Lesinurad, preferably, converting the compound 4 to Lesinurad by hydrolysis;

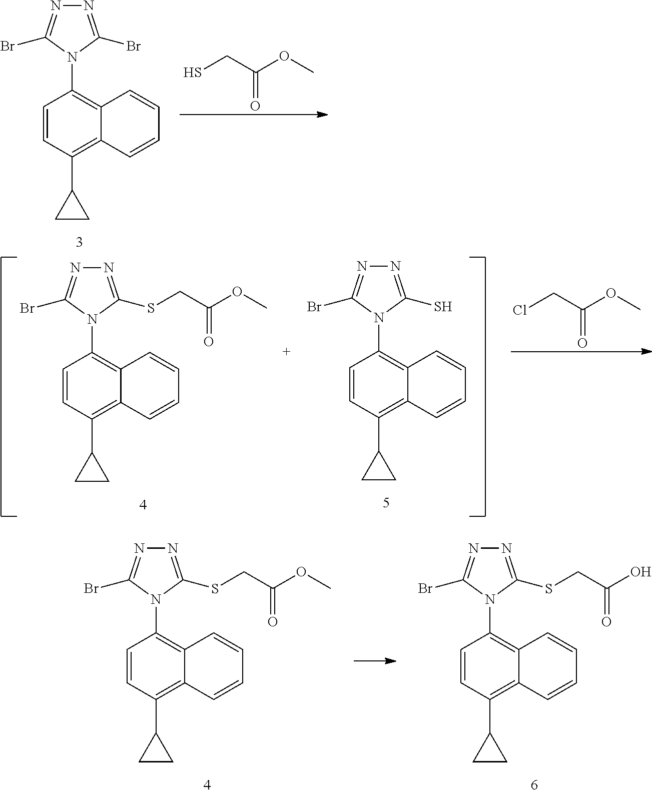

10. The preparation method according to claim 9, wherein the compound 4 obtained in the step 2) is subjected to a next reaction directly without purification and is converted into Lesinurad.

11. The preparation method according to claim 1, wherein R represents cyclopropyl.

12. The Lesinurad intermediate compound according to claim 4, wherein R represents cyclopropyl.

13. The preparation method according to claim 9, wherein the compound 4 is converted to Lesinurad by hydrolysis.

14. The preparation method according to claim 9, wherein the first solvent is selected from the group consisting of N,N-dimethylformamide, N-methylpyrrolidone and acetonitrile, or any combination thereof; the first base in the step 1) and the second base in the step 2) are independently selected from the group consisting of 1,8-diazabicycloundec-7-ene, diisopropylethylamine, triethylamine, potassium carbonate and sodium carbonate, or any combination thereof.
